(19) United States
(12) Patent Application Publication
(10) Pub. No.: US 2012/0165622 A1
(43) Pub. Date: Jun. 28, 2012

(54) METHOD AND SYSTEM FOR MEASURING PHYSIOLOGICAL PARAMETERS

(75) Inventors: Noelia RODRÍGUEZ IBÁÑEZ, Mollet del Valles - Barcelona (ES); Mireya FERNÁNDEZ CHIMENO, Mollet del Valles - Barcelona (ES); Juan José RAMOS CASTRO, Mollet del Valles - Barcelona (ES); Miguel Angel GARCIA GONZÁLEZ, Mollet del Valles - Barcelona (ES); Eduard MONTSENY MASIP, Barcelona (ES); Daniel BANDE MARTINEZ, Barcelona (ES)

(73) Assignee: FICO MIRRORS, S.A., Barcelona (ES)

(21) Appl. No.: 13/337,195

(22) Filed: Dec. 26, 2011

(30) Foreign Application Priority Data
Dec. 24, 2010 (ES) P201031939

(51) Int. Cl. A61B 5/0205; A61B 5/0402; A61B 5/085; A61B 5/053; A61B 5/00

(52) U.S. Cl. 600/301; 600/547; 600/536; 600/509; 600/484

(57) ABSTRACT

A method for determining at least one physiological parameter of a driver, pilot or transport of defence vehicle or system operator, comprising: a) injecting or inducing current into said individual; b) measuring in said individual an electrical parameter indicating their impedance, comprising: b1) measuring the electrical parameter with at least two pairs of injector-detector electrodes; b2) determining the impedance signal from the electrical parameter measured; b3) from amongst the impedance signals determined, identifying those with a physiological origin; and b4) selecting the pair of electrodes that produce the best quality impedance signal; and c) obtaining said physiological parameter from the best quality impedance signal.

### CROSS REFERENCE TO RELATED APPLICATIONS

This application claims priority under 35 U.S.C. §119(a) of Spanish Patent Application No. P201031939 for “Method and System for Measuring Physiological Parameters” filed on Dec. 24, 2010 in the name of Noelia RODRIGUEZ IBANEZ et al., which is hereby incorporated herein in its entirety.

### DESCRIPTION

1. Technical Field of the Invention

This invention lies within the field of systems for monitoring the state of alertness of an individual and detecting undesirable states. More specifically, it discloses a method and a system for the non-invasive measurement of a user's physiological parameters, in particular for a driver, pilot, or the operator of a transport or defence vehicle or system, via impedance measurements.

2. Background of the Invention

The measurement of electric impedance in the human body is a widely used non-invasive diagnostic technique that allows monitoring several physiological processes, from the build-up of body fat to the rejection of transplanted organs. Applied to the detection of biological signals in a vehicle it allows detecting, for example, the breathing, the heart rate, the user's posture or physiological condition, sine all these processes are associated to changes of the electric impedance of the body.

Several methods and devices are known aimed especially to the detection of drowsiness in vehicle drivers. Most of these methods and devices are based on how the subject is driving, extracting information regarding the use of the steering wheel, the vehicle leaving the lane or zigzagging. These systems have the disadvantage that they cannot differentiate between whether a certain way of driving corresponds to an abnormal state of the driver or, in contrast, to a specific condition of the road, traffic, or even if it is the normal way of driving for a particular driver.

Other documents show the combination of driving analysis systems with biomedical signals, generally related to heart rate or blood pressure. U.S. Pat. No. 6,575,902 B1 describes a system for assessing the state of vigilance of a driver using sensors measuring physiological variables associated with the driver and thus determining the relevant physiological state in order to decide whether or not the driver's vigilance state is satisfactory.

The problem of known methods and systems is that they do not manage to guarantee the detection of a desired physiological signal, regardless of the state of the individual or their way of driving.

There is therefore a need for a method that may allow determining the state of alertness of an individual in a reliable and robust manner, without the limitations of other known methods and avoiding the effect that the variability between different people has on common methods based on biomedical data analysis.

The method and system proposed obtain information on the individual's physiological variables related to the degree of tiredness or drowsiness from the measurement of the individual's electric impedance. These variables are controlled by the central nervous system and therefore represent objective and accurate information on the individual's state of alertness. The main advantage obtained over known systems is the redundancy in the acquisition of the signal and the intelligent management of such records in order to the best for the analysis.

### SUMMARY OF THE INVENTION

This invention provides a solution to the problem stated above by a method for determining at least one physiological parameter from an individual according to claim 1 and a measurement system of at least one physiological parameter according to claim 14. The dependent claims define the preferred embodiments of the invention.

A first inventive aspect defines a method for determining at least one physiological parameter of an individual comprising a) injecting or inducing current into said individual; b) measuring an electrical parameter in said individual indicating impedance, and c) obtaining said physiological parameter from the best quality impedance signal, wherein step b) includes: b1) measuring the electrical parameter with at least two pairs of electrodes; b2) determining the impedance signal from the electrical parameter measured; b3) from amongst the impedance signals determined, identifying those with a physiological origin; and b4) selecting the pair of electrodes that produce the best quality impedance signal.

The physiological parameter to be determined in the individual is preferably at least one of the following: respiration, heart rate, posture, state of alertness (drowsiness, vigilance, fatigue, etc.).

In one embodiment the electrical parameter measured is the variation in voltage between one pair of electrodes and the impedance signal is calculated as the ratio between the voltage measurement and the current injected.

In one embodiment the selection of the pair of electrodes that produce the best quality impedance signal comprises calculating, for each pair of electrodes associated to a physiological impedance signal, a parameter indicating the quality of the measurement, preferably the signal/noise ratio.

In one embodiment the method of the invention comprises determining the individual's respiratory signal and processing said respiratory signal to detect the individual's state of alertness.

In one embodiment the method of the invention comprises self-scaling the impedance signal measured with each pair of electrodes. The main advantage of the self-scaling is the correction of factors that are difficult to control, such as the impedance of the electrodes and the drift in the capture system, amongst others.

A second inventive aspect defines a system for determining at least one physiological parameter of a driver, pilot or transport of defence vehicle or system operator comprising:

means for injecting current in order to inject or induce current in said individual; detection means to measure an electrical parameter indicating the impedance in said individual; and

signal processing means configured to determine and process an impedance signal from an electrical parameter measured by the sensing means; and data processing means configured to
identify the impedance signals with a physiological origin,
select the injector-detector pair that produces the measurement with the best quality, and
obtaining a physiological parameter from the impedance signal.

The data processing means are configured to search amongst all impedance channels those that show a cause for variation of the impedance associated to a physiological system, such as the respiratory system, the heart system, etc.

The system of the invention decides which is the most favourable pair of electrodes, i.e. the pair of electrodes that offers the best quality measurement in order to obtain the physiological signal. The electronic system is configured to provide the individual's electric impedance from the current injected and the electrical parameter measured, usually the voltage, from the best pair of electrodes, in order to guarantee the quality of the signal used for the analysis.

Preferably, the current injection means and the detection means are arranged on the steering wheel or controls of the transport or defence vehicle or system and/or on the seat of the driver, pilot or operator thereof. Advantageously, this method does not require the cooperation of the individual being measured, in the sense of placing electrodes or other type of sensors on their body.

One embodiment of the system of the invention comprises one or several respiration and/or movement sensors, preferably one or several sensors integrated into different parts of the seat in order to detect and identify the type of movement of the individual and/or at least one Doppler radar.

In one embodiment the current injection means and the detection means are arranged such that the thoracic cavity lies in the path of the current driven through the individual. This allows maximising the variations of impedance with respiration.

The method and system of the invention allow obtaining information on the individual's physiological variables related to the degree of tiredness or drowsiness from the measurement of the individual's electric impedance.

The method of the invention can be implemented in an electronic control unit of the vehicle, to which the system's electrodes would be connected.

### BRIEF DESCRIPTION OF THE DRAWINGS

In order to complete the description made below and to aid towards a better understanding of the characteristics of the invention, the present descriptive memory is accompanied by a set of drawings showing, for purposes of illustration only and in a non-limiting sense:

FIGS. 1 and 2 show diagrams for the steps of the measurement and determination of the system's impedance signal and the method of the invention.

FIG. 3 shows a diagram of an embodiment of the method of the invention.

FIG. 4 shows a possible configuration for arranging electrodes on the steering wheel and in the seat of the vehicle.

FIG. 5 shows an example of a respiration signal obtained by the method of the invention.

### EMBODIMENTS OF THE INVENTION

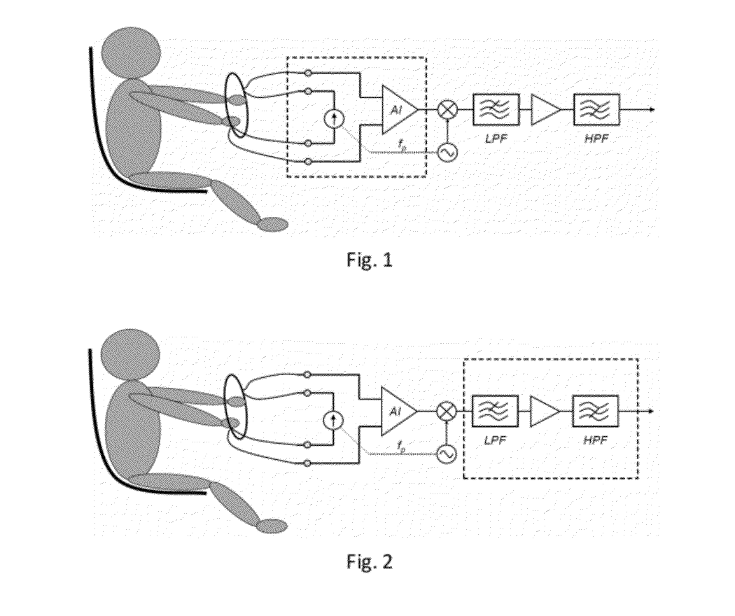

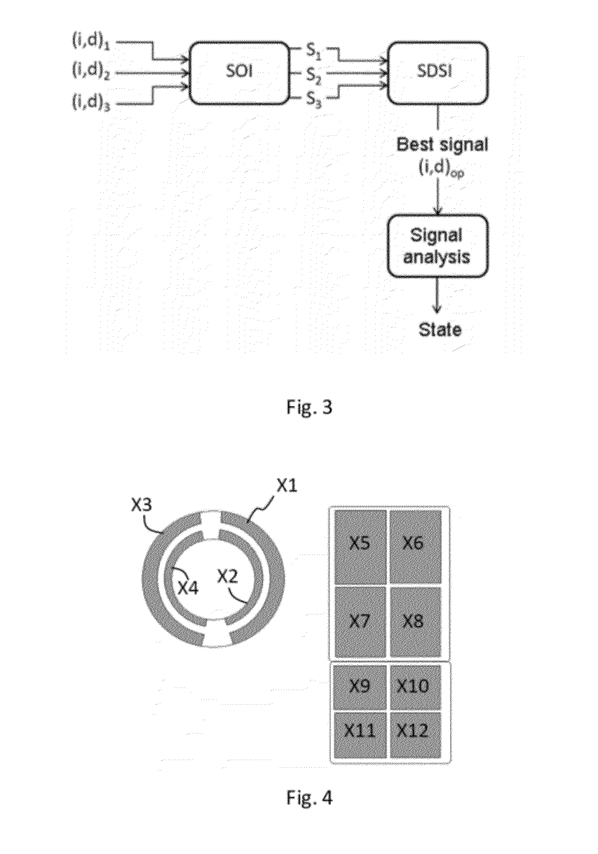

Described below are the steps of the method according to a preferred embodiment of the invention, which is diagrammatically represented in FIG. 3. First of all we inject or induce via the electrodes an electrical current into the body of the individual with variable amplitude and sufficient to obtain a suitable sensitivity of the measurements, respecting the maximum levels established in the safety standard for medical equipment with applied BF parts (EN 60601-1:2006). The current generated at the injection electrodes passes through the individual's body and reaches the detection electrodes. The electrodes must be in contact with the individual, either in direct contact with their skin or with their clothes. Measured below is the variation in voltage for each pair of injector-detector electrodes ((i,d)₁, (i,d)₂, (i,d)₃) involved in the measurement, and the impedance signal and the ratio between the voltage measured and the current injected is calculated. The physiological parameters of interest shall modulate the individual's impedance over time. This step of the process is represented diagrammatically in the section indicated with a box in FIG. 1.

Since the variations in impedance associated to the biological processes are low, the measurement cannot be taken directly, since the impedance of the electrodes themselves would mask the impedance to be measured. To avoid this effect we use a 4-wire impedance measurement system.

The signals of each injector-receptor pair ((i,d)₁, (i,d)₂, (i,d)₃) are processed via signal processing means (SOI) that are configured to determine and process an impedance signal from the values of the current injected and the voltage measured by the detection electrodes, as shown diagrammatically in the step indicated with box in FIG. 2. The processing of the impedance signal may include: a) a step of pre-processing the impedance signal, preferably including a band-pass filter, b) signal amplification and c) a signal post-processing step preferably comprising a coherent demodulation and a filtering step. This may additionally include an optional step of self-scaling the impedance signal.

The processed impedance signals (S₁, S₂, S₃) pass to data processing means (SDSI) configured to differentiate the impedance signals with a physiological origin from those that do not have a physiological origin and to select the pair of injector-detector electrodes ((i,d)ₒₚ) with the best quality measurement (Sₒₚ). In a preferred embodiment the first data processing means (SDSI) perform the following steps: a) a self-scaling of the signal (S₁, S₂, S₃), if not previously performed by the signal processing means (SOI) in the previous step, b) calculating the signal/noise ratio (SNR) or another parameter indicating the quality of the signal, c) selecting as the best signal (Sₒₚ) the signal with the greatest signal/noise ratio (SNR) or the signal with the best quality according to the quality parameter used if using a different quality parameter, and d) selecting the injector-detector pair ((i,d)ₒₚ) that has registered the optimal signal as the best electrodes for recording.

The signal (Sₒₚ) selected as optimal for the analysis passes to a signal analysis module (SA) comprising data processing means configured to analyse the impedance signal and to calculate a physiological parameter from it. Preferably, the signal/noise ratio (SNR) or the alternative quality parameter is regularly evaluated and, if detected to fall below a certain acceptance threshold, indicating that the signal is losing quality, the selection process would be repeated in order to select a new signal and a new pair of optimal injector-detector electrodes ((i,dₒₚ).

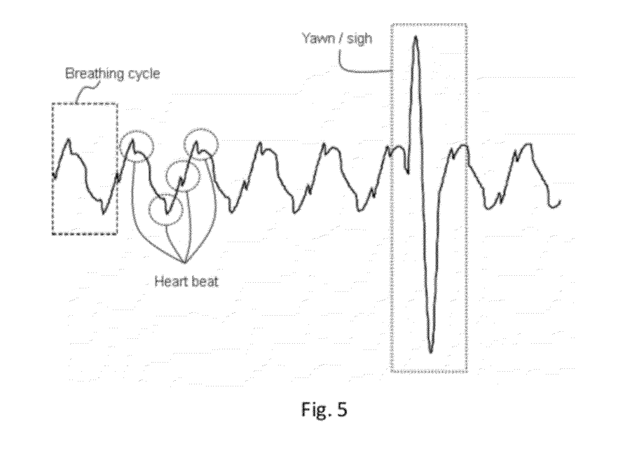

The physiological parameter to be determined may be, for example, a physiological condition of the driver, such as the degree of drowsiness. In the example of three pairs of injector-detector electrodes ((i,d)₁, (i,d)₂, (i,d)₃) we obtain the impedance signal for each pair (S₁, S₂, S₃) and select the best signal Sₒₚ to determine the degree of drowsiness of the driver. By analysing the signal Sₒₚ we can determine the individual's condition. In order to detect drowsiness, for example, we can extract from the impedance the individual's respiratory signal in order to recognise therein features indicating the individual's degree of drowsiness. FIG. 5 shows the respiratory signal and the features corresponding to a respiratory cycle, to a heart beat and to yawning or sighing. According to the features identified in the signal, we can determine, for example, whether the driver is alert, tired or drowsy.

Preferably, the current injection means include an electronic system for generating current at one or several frequencies, with variable amplitude, meeting the safety requirements for medical equipment with applied BF parts stated in Standard EN 60601-1: 2006 and two or more electrodes of any size for current injection (or current induction, in the case of induction sensors).

The electrodes can be located in one or several points of the cabin of the transport, defence or control system, in contact with the user's skin or clothes. The electrodes can be made of any kind of conductive material with a maximum resistivity of 10 Ohms/cm. The electrodes must be separated from one another by insulating material. The pairs of current injection electrodes can be multiplexed over time and/or frequency.

Preferably, the detection means for measuring electrical voltage in the individual include two or more electrodes of any size to detect voltage and an electronic system to measure the voltage between one or more pairs of electrodes.

The electrodes will be located at one or several points of the cabin of the transport, defence or control system in contact with the user's skin or clothes and can be made of any type of conductive material with a maximum resistivity of 10 Ohms/cm. The electrodes must be separated from one another by insulating material. All pairs of electrodes for measuring voltage can be multiplexed over time and/or frequency in order to select only those in which there is a sufficiently low coupling impedance with the user. For vehicles, the voltage measuring electrodes will be preferably located on the steering wheel and the seat, although this configuration is not exclusive.

The electrodes of the system can operate indistinctly and interchangeably as both injection or detection electrodes, under the condition that not more than one injector operates at the same time. The size of the electrodes can be from nanometric to the maximum allowed by the cabin design.

The signal processing means (SOI) for obtaining a physiological signal of interest from the individual's impedance preferably comprise an electronic system for extracting the physiological signal of interest, formed by a signal pre-processing step, an amplifier and a signal post-processing step.

The signal pre-processing step preferably includes a pass-band filtering system. The amplification step preferably comprises an instrumentation amplifier. The signal post-processing step preferably includes a coherent demodulator and a filtering step or any other equivalent configuration that performs the same functions.

Preferably, the system comprises suitable means for controlling the measurement process, such as an electronic control system based mainly, but not exclusively, on a microprocessor or microcontroller.

In a preferred embodiment the seat electrodes are performed with a polyester tissue mixed at 50% with stainless steel, its resistance being 1 Ohm/cm. The cover for the steering wheel is woven with a polyester thread coated with a copper and silver layer, with a surface layer of silver. Advantageously, this material is somewhat elastic due to the way in which it is woven. The electric resistance of this tissue is very low (0.3 Ohms/cm) and since the surface layer is of silver, it can be considered biocompatible.

FIG. 4 shows an example of a possible configuration of electrodes (X1-X12) on the steering wheel and seat of a vehicle, wherein the electrodes have been represented in grey and the white areas correspond to insulating tissues. This configuration of electrodes allows considering three measurement configurations: steering wheel-steering wheel, steering wheel-seat and seat-seat. According to the method of the invention, a current is injected from any of the electrodes in the system (which would then be an injector) and the level of the signal obtained in the other electrodes, acting as receptors, is assessed. The best configuration for capturing the biomedical signal is chosen from amongst the following configurations:

Steering wheel-steering wheel configuration: This requires the individual to have both hands on the steering wheel. In this case, the injector would be one of the 4 electrodes located on the steering wheel and the receptors would be any of the other two located on the opposite side (in order to have an optimal signal level) or on the same side of the steering wheel as the one acting as an injector. This would be the best configuration for correct driving.

Steering wheel-seat configuration: This requires at least one hand to be in contact with the electrodes on the steering wheel. In this case one of the electrodes on the steering wheel that is in contact with the subject would act as an injector and as a receptor any of the other electrodes in the system, taking into account that the best configuration would be that the receptors were located in the backrest area opposite the area of the steering wheel that is in contact with the hands. This would be the best configuration for driving with one hand.

Seat-seat configuration: This requires the driver to be well seated. Any electrode in the seat acts as an injector and any electrode in the seat located on the opposite side of the seat acts as a receptor. This would be the best configuration for driving with no hands.

By alternating the different measurement configurations, steering wheel-steering wheel, steering wheel-seat and seat-seat we can always guarantee the detection of the physiological signal regardless of the state of the individual or their way of driving.

1. A method for determining at least one physiological parameter of a driver, pilot or transport of defence vehicle or system operator comprising the following steps:
a) injecting or inducing current into said individual;
b) measuring an electrical parameter in said individual indicating their impedance, comprising
b1) measuring the electrical parameter with at least two pairs of injection-detection electrodes;
b2) determining the impedance signal from the electrical parameter measured;
b3) from amongst the impedance signals determined, identifying those with a physiological origin; and
b4) selecting the pair of electrodes that produce the best quality impedance signal;
c) obtaining said physiological parameter from the impedance signal with the best quality.

2. A method for determining at least one physiological parameter according to claim 1, wherein the electrical parameter measured is the variation of voltage between a pair of electrodes and the impedance signal is calculated as the ratio between the voltage measured and the current injected.

3. A method for determining at least one physiological parameter according to claim 1, comprising:
pre-processing the signal,
amplifying the signal, and
post-processing the signal.

4. A method for determining at least one physiological parameter according to claim 3, wherein the pre-processing of the signal comprises a band-pass filter.

5. A method for determining at least one physiological parameter according to claim 3, wherein the post-processing of the signal comprises a coherent demodulation and a filtering step.

6. A method for determining at least one physiological parameter according to claim 1, wherein the selection of the pair of electrodes that produce the impedance signal with the best quality comprises calculating, for every pair of electrodes associated to a physiological impedance signal, a parameter indicating the quality of the measurement.

7. A method for determining at least one physiological parameter according to claim 1, wherein the parameter indicating the quality of the measurement performed by each pair of electrodes is calculated as the signal/noise ratio.

8. A method for determining at least one physiological parameter according to claim 1, comprising self-scaling the impedance signal measured with each pair of electrodes.

9. A method for determining at least one physiological parameter according to claim 1, wherein the parameter indicating the quality of the signal is assessed regularly, and if it falls below a certain acceptance threshold, step b) is repeated in order to select a new optimal pair of electrodes.

10. A method for determining at least one physiological parameter according to claim 1, wherein there are electrodes arranged on the steering wheel or commands of the transport or defence vehicle or system and/or on the seat of the driver, pilot or operator thereof.

11. A method for determining at least one physiological parameter according to claim 1, comprising analysing signals by at least one breath and/or movement sensor and/or an additional vehicle bus signal analysis system.

12. A method for determining at least one physiological parameter according to claim 1, wherein the physiological parameter determined is a heart signal and/or a breath signal and/or the individual's heart rate and range and/or respiratory rate and range.

13. A method for determining at least one physiological parameter according to claim 1, comprising determining the individual's respiratory signal and processing said respiratory signal in order to detect the individual's state of alertness.

14. A system for determining at least one physiological parameter of a driver, pilot or transport of defence vehicle or system operator comprising:
means for injecting current in order to inject or induce current in said individual;
detection means to measure an electrical parameter indicating the impedance in said individual; and
signal processing means configured to determine and process an impedance signal from an electrical parameter measured by the sensing means; and
data processing means configured to
identify the impedance signals with a physiological origin,
select the injector-detector pair that produces the measurement with the best quality, and
obtaining a physiological parameter from the impedance signal.

15. A system for measuring at least one physiological parameter according to claim 14, wherein the current injection means an the detector means are electrodes arranged on the steering wheel or the controls of the transport or defence vehicle or system and/or on the seat of the driver, pilot or operator thereof.

16. A system for measuring at least one physiological parameter according to claim 14, comprising one or several respiration and/or movement sensors.

17. A system for measuring at least one physiological parameter according to claim 16, comprising a sensor integrated in the seat to detect movement of the individual.

18. A system for measuring at least one physiological parameter according to claim 17, comprising a plurality of sensors integrated into different parts of the seat in order to identify the type of movement.

19. A system for measuring at least one physiological parameter according to claim 16, comprising at least one Doppler radar to measure movements of the driver's or operator's thoracic cavity.

20. a system for measuring at least one physiological parameter according to claim 14, wherein the physiological parameter is related to respiration and wherein the current injection means and the detection means are arranged such that the thoracic cavity lies in the path of the current.

21. A system for measuring at least one physiological parameter according to claim 14, wherein the means for injecting current and the means for measuring an electrical parameter are formed by low impedance electrodes.

22. A system for measuring at least one physiological parameter according to claim 14, comprising a high impedance source of output current for injecting the current into the individual.

23. A system for measuring at least one physiological parameter according to claim 14, comprising a circuit for measuring high input impedance voltage.
